(19) United States
(12) Patent Application Publication
(10) Pub. No.: US 2016/0324745 A1
(43) Pub. Date: Nov. 10, 2016

(54) STABILIZED PHYCOCYANIN FOR BLUE COLOR

(71) Applicant: BASF SE, Ludwigshafen (DE)

(72) Inventors: Thrandur HELGASON, Mannheim (DE); Heribert BOHN, Wattenheim (DE); Anja WEILAND, Maxdorf (DE); Christian SOWA, Neustadt (DE); Thomas GOTTSCHALK, Shanghai (CN)

(21) Appl. No.: 15/104,737

(22) PCT Filed: Oct. 28, 2014
(86) PCT No.: PCT/EP2014/073057

§ 371 Date: Jun. 15, 2016

(30) Foreign Application Priority Data
Dec. 18, 2013 (EP) 13197910.6

(51) Int. Cl. A61K 8/49; A23L 2/58; A23L 5/46; C07K 14/195; A61K 8/60; A61Q 19/00; A61K 47/26; A61K 47/22; A61K 47/42; A61Q 11/00; A61Q 1/02; A23K 20/179; A61K 8/64

(52) U.S. Cl. CPC A61K 8/4913; A23K 20/179; A23L 2/58; A23L 5/46; C07K 14/195; A61K 8/602; A61K 8/645; A61K 47/26; A61K 47/22; A61K 47/42; A61Q 11/00; A61Q 1/02; A61Q 19/00; A23V 2002/00; A61K 2800/43; A61K 2800/10

(57) ABSTRACT

The present invention relates to blue coloring composition useful in the manufacture of food, feed, cosmetic and pharmaceutical products and preparations based on a stabilized phycocyanin, which is a complex of at least one phycocyanobilin and at least one polyphenol as well as a process for the formation of this complex.

### CROSS-REFERENCE TO RELATED APPLICATIONS

This application is a national stage application (under 35 U.S.C. §371) of PCT/EP2014/073057, filed Oct. 28, 2014, which claims benefit of European Application No. 13197910.6, filed Dec. 18, 2013, both of which are incorporated herein by reference in their entirety.

The present invention relates to blue coloring composition useful in the manufacture of food, feed, cosmetic and pharmaceutical products and preparations based on a stabilized phycocyanin, which is a complex of at least one phycocyanobilin and at least one polyphenol as well as a process for the formation of this complex.

Phycocyanin is a pigment protein complex with a characteristic light blue color, absorbing orange and red light near 620 nm. Phycocyanin are found in Cyanobacteria, previously called blue green algae. The pigment, respectively the chromophore of phycocyanin is phycocyanobilin. This chromophore is a tetrapyrrol which is covalently bound to the protein by a thioether bond. Additionally to the thioether bond the chromophore interacts with the protein by hydrogen bonding which results in favorable conformation of the chromophore. Intern this results in strong blue color. Phycocyanobilin can also be found in allophycocyanin, phycoerythrin, and other pigment proteins.

Phycocyanin is commonly isolated from Spirulina algae and shows many dietary and therapeutic attributes. Therefore Spirulina and Spirulina extracts have been used for long time as food or nutritional components. One method of preparing such components is disclosed in WO 03/080811 A1 which describe a way-out of the repugnant apparent due to the deep blue color by denaturating the chromoprotein by heating to 70 to 100° C. Accordingly Phycocyanin is very sensitive to temperature and pH-changes in the environment because of its polypeptide subunits. (Seo et al., Int. J. Mol. Sci. 2013, 14, 1778-1787). On the other side any changes to the protein-chromophore interaction leads to loss of color.

Also the use of Spirulina extracts or phycocyanin in food, especially beverage, is already known (for example CN 103 054 117 A) there is still a need for an nontoxic, innoxious blue colorant in food, feed, cosmetic or pharmaceutical preparations which fulfills all the high safety standards as required by the FDA or the European Community. Additionally the blue color has to be stable over a long period of time as well as at low, acidic pH and well as high temperature of about 60-130° C., as there are used for example in the pasteurization process. The chromophore has furthermore to be stabilized against oxidation which also would reduce the color.

The color, e.g. the colorant (color compound) has to be stable especially at high light condition at room temperature. Additionally a precipitation of the colorant composition, especially in beverage has to be avoided because this would lead to an increased turbidity and sedimentation.

Therefore the stabilization of color compounds that relies on specific protein structure for the correct color is very difficult.

Furthermore the color compound, especially a complex of the chromophore with another compound, whose color is based on the interaction of the chromophore with this other compound, has to be inert regarding the reaction or interaction with other molecules which would lead in a loss of color in the final preparation like food, feed, cosmetic and pharmaceutical.

Accordingly complex of at least one phycocyanobilin and at least one polyphenol obtainable by mixing the polyphenol with a composition comprising at least one phycocyanobilin in an aqueous solution has been found which fulfills all the requirements.

In one embodiment the complex of at least one phycocyanobilin and at least one polyphenol is obtained by mixing the polyphenol with a composition comprising at least one phycocyanobilin in an aqueous solution.

In one embodiment of the invention the composition comprising at least one phycocyanobilin comprises a phycocyanin-fragment consisting of at least one phycocyanobilin and at least one amino acid. The amino acid can interact with the chromophore by hydrogen bonding and/or by a thioether bond. The compound may comprise further amino acids which interacts by hydrogen bonding with the phycocyanobilin.

In one alternative this complex is a stable complex.

“Stable” means, that a complex has a low, preferably very low dissociation constant due to a high thermodynamic and/or kinetic stability so that a chemical equilibrium is shifted to the side of the complex. Therefore the complex can be soluble in water but does not dissociate.

In one embodiment the complex is stable at pH 1 to 8, preferably 1.5 to 7, pH 2 to 5, more preferably 2.5 to 4, pH 3 to 4, especially pH 3.5.

“Stable” means that the complex maintains a blue color, preferably blue pure or blue violet, having an maximum of absorption of light with a wavelength within an interval from 550 to 670 nm, preferably 560 to 640 nm, more preferably 580 to 620 nm, especially from 600 to 610 nm.

In one alternative there are also two peeks possible within any of the above mentioned range.

In a further embodiment the complex of the invention shows an increased absorption of light with a wavelength within the interval from 550 to 670 nm, preferably 560 to 640 nm, more preferably 580 to 620 nm, especially from 590 to 610 nm.

The increased absorption of light with a wavelength within the above mentioned interval is from 5% to 200%, or more than the absorbance of phycocyanin preferably from 5% to 95%, from 10% to 90%, from 20% to 90%, from 25% to 90%, more preferably from 25% to 80%.

The ratio of phycocyanin to polyphenol is 1:10 to 10:1, preferably 1:2 to 5:1, more preferably 1:1 to 2:1, especially 1:1.

In one embodiment the composition comprising at least one phycocyanobilin is obtainable or is obtained by cleaving phycocyanin.

The isolation of phycocyanin from Spirulina algae is well known in the art and can be performed for example according to the method disclosed by Seo et al. (Int. J. Mol. Sci. 2013, 14, 1778-1787), Muthulakshni et al. (J. Alga Biomus Utln. 2012, 3, 7-11), Hemlata et al. (J. Alga Biomus Utln. 2011, 2, (1), 30-51) or Gantar et al. (J. Biotechnol. 2012, 159 (1-2), 21-26). Also commercially available phycocyanin can be used, as for example commercial powder from DIC called Linablue G1.

In one embodiment the polyphenol of the invention is selected from the group of compounds comprising a least two phenol rings, each of them substituted with at least two hydroxy-groups, preferably three hydroxy-groups and/or the polyphenol comprises a least one carboxylate ester group and/or carboxylic acid group.

According to the invention the term “carboxylic acid group” encompasses also a carboxylate-group.

In one alternative the polyphenols of the invention comprises esters or polymers of gallic acid.

The polyphenol compounds can comprise in addition to the phenol ring with at least two hydroxy-groups linear saturated or unsaturated alkyls, preferably C2 or C3 alkyls having a carboxylate group. In the polyphenol this carboxylate group is used for the ester bonding or is a free carboxylic acid or carboxylate-group.

The polyphenol can also comprise cycloalkyls or hetero-cycloalkyls with a carbon chain of C5 or C6. Preferably glucose or other sugar are used.

In one embodiment the polyphenol of the invention is selected from the group of rosmarinic acid, tannic acid, digallic acid, condensed tannins (condensation products of flavans), quercitannic acid, gallotannic acid, quercitin, ellagitannins, castalagin, castalin, casuariticin, grandinin, punicaligin, punicalin, roburin A, tellimagrandin II, terflavin B, vescaligin, pendunculagin, casuariin, castlin, vescalin, preferably rosmarinic acid, tannic acid, digallic acid, condensed tannins, quercitannic acid, gallotannic acid, ellagitannin, more preferably tannic acid (CAS 1401-55-4).

A further subject of the present invention is the complex of at least one phycocyanobilin and at least one polyphenol obtainable by mixing the polyphenol with a composition comprising at least one phycocyanobilin in an aqueous solution comprising a protein which has a higher isoelectrical point than the phycocyanin

### A BRIEF DESCRIPTION OF THE FIGURE

FIG. 1 Illustrates the color strengthen of the complex according to the invention.

The protein with the higher isoelectrical point is highly soluble in an aqueous solution, preferably an acidic aqueous solution with a pH 1 to 7, preferably 1.5 to 7, pH 2 to 5, more preferably 2.5 to 4, pH 3 to 4, especially pH 3.5; more preferable in a beverage.

In one alternative the protein with a higher isoelectrical point is selected from animal proteins, plant proteins, proteins from microorganism, preferably selected from the group consisting of whey protein isolate, soy protein, polylysine.

In further alternative the protein has a isoelectrical point of 4-9.

In one alternative of the invention water soluble polymer is used instead of the protein with higher isoelectrical point.

A further object of the invention is a process for formation of a complex comprising the step of mixing a polyphenol with a composition comprising at least one phycocyanobilin in an aqueous solution.

In one embodiment the composition comprising at least one phycocyanobilin is obtained by cleaving phycocyanin by chemical and/or enzymatic cleavage.

In one alternative phycocyanin is cleaved by proteolysis.

The proteolysis can be carried out by action at least one strong acid and optionally heat.

The strong acid with a pKa of −1 and below can be for example HCl, formic acid, H2SO4, HNO3 or mixtures thereof, preferably HCL.

Optionally the mixture of strong acid and phycocyanin is heated to a temperature of 20-100° C. Optionally the acid can be in the form of ion-exchange resin.

In one embodiment phycocyanin powder is added to the concentrated strongacid, preferably HCL and stirred.

The cleavage of phycocyanin leads to phycocyanin-fragments. Some of the fragments comprises the phycocyanobilin.

The cleaving reaction is stopped by dilution with water. This step also results in precipitation of the phycocyanin fragments.

In one alternative polyphenol is added for a better precipitation.

The solution is filtered to remove the strong acid resulting in a phycocyanin-fragment filter-cake.

The filter-cake is then redissolved in pure water and stirred or mixed until all phycocyanin-fragments are dissolved. The solution is spray-dried resulting in fine powder with good solubility in water.

The complex of the invention is formed by mixing a polyphenol with a composition comprising at least one phycocyanobilin in an aqueous solution.

In a further embodiment an additional protein is added to the complex of the invention, whereby this additional protein has a higher isoelectrical point than phycocyanin

Subject matter of the present invention is also a complex of at least one phycocyanobilin and at least one polyphenol and an additional protein, (preferably with a higher isoelectrical point), or polymer. This second complex of the invention is stable in solution with a pH (preferable a pH of 3-4) which is very closed to the isoelectrical point of the phycocyanin.

The protein with higher isoelectrical point is selected from animal protein, plant proteins, preferably selected from the group consisting of whey protein isolate, soy protein, polylysine.

The complex of the present invention as well as the second complex of the invention can be used as colorants.

A further subject of the invention is the use of the complex of the invention as well as the second complex in food, feed, cosmetic or pharmaceutical preparations.

Subject of the invention is also any product produced according to any of the above described processes.

A further subject of the invention is also the use of the complexes of the above mentioned complexes as colorants.

An additional subject matter of the invention is the product of the invention, namely food, feed, cosmetic and pharmaceutical preparations comprising the complex of the invention or the second complex of the invention, preferably as colorant.

In one embodiment food is selected from the group comprising beverage, beverages like soft drinks, flavoured water, fruit juices, punches or concentrated forms of these beverages but also alcoholic beverages and instant beverage powders, ice-cream, cake, drops, cheese, milk product like milk drinks or yoghurt, soy milk and the like, confectionary products, gums, dessert, candies, puddings, jellies, instant pudding powder, but also in snacks, cookies, sauces, cereals, salad dressing, soups.

In one embodiment cosmetic preparations are selected from cream, tooth paste, makeup, dermal products.

In one embodiment pharmaceutical preparations are selected from unguents, pills, tablets, capsules.

It was surprising and could not expected by a person skilled in the art that the objects underlying the present invention could be solved by the complex, the process or other subject matter of the invention. It was particularly surprising that the complex of the invention is stable in an aqueous solution during pasteurization, especially at 90° C. for 15 minutes and additionally during storage in intense light exposure, as seen during storage of beverage on the store shelf for up to 6 months.

It was further surprising that the complex of the invention is stable at low pH, preferably 1 to 8, preferably 1.5 to 7, pH 2 to 5, more preferably 2.5 to 4, pH 3 to 4, especially pH 3.5.

Furthermore, the complex of the present invention and especially the second complex of the invention show no precipitation and no increase in turbidity in aqueous composition, especially beverages.

As colorant, preferably in food and especially in beverage, the complex of the present invention respectively the second complex of the present invention are used in an amount of: 1-5000 ppm, preferably 10-700 ppm, more preferably 10-500 ppm, especially 50-400 ppm,

A beverage of the present invention can additionally comprise:

In one embodiment the beverage is clear or turbid with NTU from 1-500.

In one embodiment color of the product can be changed from blue to green by adding a carotenoid, or any other yellow food color. Therefore the product of the present invention comprises carotenoid optionally melt and/or solved and/or isomerized from trans to cis in triacylglycerol oil, such as MCT oil (medium-chain triacylglycerol), olive oil, corn oil, sunflower oil, peanut oil, soy oil or other alternative vegetable oil, preferably MCT oil.

On one embodiment the product of the beverage comprises and oil soluble antioxidant.

In a further embodiment the product comprises a carbohydrate selected from the group comprising: mono-, di- and oligosaccharides, glucose syrup, maltose and trehalose, preferably glucose syrup, maltose and trehalose. The saccharides contains glucose, fructose, galactose or mannose.

The product of the present invention comprise in one alternative at least one water-soluble antioxidant selected from the group consisting of:
natural compounds that are active as antioxidants because they comprise a phenolic OH-group in their chemical structure: like hydroxy derivatives of cinnamic acid, e.g. hydroxycinnamic acids, hydroxycinnamates, which are a class of polyphenols having a C6-C3 skeleton, for example hydroxyhydrocinnamate;
caffeic acid, ferulic acid, tyrosol, hydroxytyrosol, cinnamic acid, chlorogenic acid, coumarin, coumarinic acid, sinapic acid, cinnamic acid, chicoric acid, and esters of any of these compounds with C1-C20;
extracts of plants rich in at least one of the above compounds;
rosmarinic acid, hydroxytyrosol;
extracts from common spices. In one embodiment common spices are selected from the group comprising rosemary, lemon balm, oregano, thyme, peppermint, sage or similar plants comprising or being rich in at least one of the above compounds;
flavons, which are a class of natural compounds of which more than 5000 exist, used as antioxidants can be any of them as extracted from plants such as tea or any other plant that comprise or is rich in catechin or epicatechin or derivatives, whereby these compounds can be glycosylated with carbohydrates or esterified with fatty acids C1-C20 or gallic acid; extracts from plants such as tea, olives, pears, apples comprising or being rich in one or more of the above mentioned compounds;
sodium ascorbate, polyphenole, Teanova 80, glutathione, lipoic acid, catechin, punicalagin, xanthone, benzotropolones, preferably sodium ascorbate.

### EXAMPLES

### Example 1

### Formation of Cleaved Phycocyanin Powder

18 g of Phycocyanin (commercial powder from DIC called Linablue G1) was added into 60 g of concentrated HCl. This solution was stirred for 2 hours. This results in partial breakdown of the protein. After 2 hours the reaction was stopped by pouring the HCl/phycocyanin solution into 533 g of water. The dilution into water, results in precipitation of the phycocyanin allowing for filtration or centrifugation to separate the cleaved-phycocyanin out. The cleaved phycocyanin is then re-dispersed in water by ball mill. The cleaved phycocyanin solution is then dried by spray drying resulting in water soluble powder.

### Example 2

### Storage Stability

Samples were stored at 500 ppm concentration in a sealed glass vial at room temperature (22° C.). Vials where placed in a straight row 30 cm from the light source, thus exposing all samples to the same amount of light 7000 LUX. Color intensity was then measured by measuring the absorbance at maximum absorption in a spectrometer (UV-vis spectroscopy HP 8452A). This test is made to simulate the storage of beverages directly under the lighting source of a supermarket shelf; however the light intensity is substantially higher than the supermarket shelf in order to evaluate color stability to light in an accelerated fashion. 7 days of storage in this test translates roughly to 3 months of storage under regular store lighting.

All calculation of color loss are based on absorption of the final mixture in the beginning of the trial (day 0) to the end of the investigation, using the following formula:

((Abs_{beginning}−Abs_{end})/Abs_{beginning})*100

Where Abs means absorption selected at the wavelength where maximum absorbance was measured. Samples where measured directly in 500 ppm aqueous solutions.

### Example 3

Uncleaved phycocyanin (commercial powder from DIC called Linablue G1) powder was dissolved in water at pH 2 and 500 ppm concentration then the vial was sealed and placed in accelerated storage for 5 days. After the storage period the absorption was measured.

This sample lost 100% of its color strength.

### Example 4

Cleaved phycocyanin (see example 1) (commercial powder from DIC called Linablue G1) was dissolved in water at pH 2 and 500 ppm concentration then the vial was sealed and placed in accelerated storage for 5 days. After the storage period the absorption was measured.

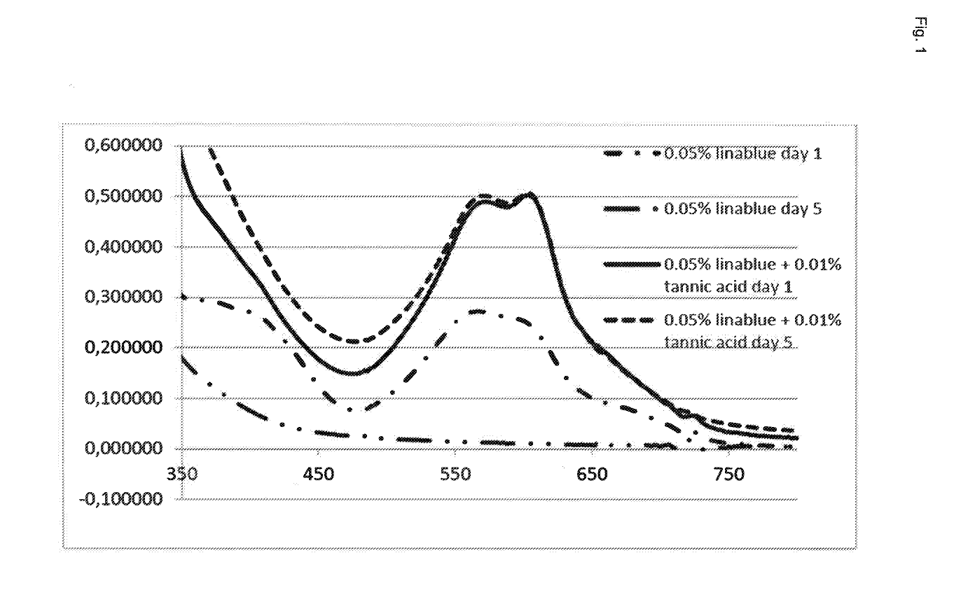

This sample lost 100% of its color strength (see FIG. 1).

### Example 5

Uncleaved phycocyanin powder (commercial powder from DIC called Linablue G1) was dissolved in water at pH 3.5 and 500 ppm concentration then the vial was sealed and placed in accelerated storage for 5 days. After the storage period the absorption was measured.

This sample lost 100% of its color strength, furthermore substantial aggregation was visible.

### Example 6

Cleaved phycocyanin (see example 1) (commercial powder from DIC called Linablue G1) was dissolved in water at pH 3.5 and 500 ppm concentration then the vial was sealed and placed in accelerated storage for 5 days. After the storage period the absorption was measured.

This sample lost 100% of its color strength, no aggregation was visible.

### Example 7

Uncleaved phycocyanin powder (commercial powder from DIC called Linablue G1) was dissolved in water at pH 2 and 500 ppm concentration, then 100 ppm of tannic acid was added to the solution, thus forming a complex.

The complex formation was indicated by a small increase in absorption of solution (6.4% increase in absorption and a shift in absorption maximum from 628 nm to 632 nm).

Finally the vial was sealed and placed in accelerated storage for 5 days. After the storage period the absorption was measured.

This sample lost 64% of its color strength.

### Example 8

Cleaved phycocyanin (see example 1) (commercial powder from DIC called Linablue G1) was dissolved in water at pH 2 and 500 ppm concentration, then 100 ppm of tannic acid was added to the solution, thus forming an complex

The complex formation was indicated by increase in absorption of solution (18% increase in absorption and a shift in absorption maximum from 598 nm to 604 nm). Finally the vial was then sealed and placed in accelerated storage for 5 days. After the storage period the absorption was measured.

This sample lost 35% of its color strength.

### Example 9

Cleaved phycocyanin (see example 1) (commercial powder from DIC called Linablue G1) was dissolved in water at pH 2 and 500 ppm concentration, then 500 ppm of gallic acid was added to the solution, however no increase in absorption was measured. Finally the vial was then sealed and placed in accelerated storage for 5 days. After the storage period the absorption was measured.

This sample lost 100% of its color strength.

### Example 10

Cleaved phycocyanin (see example 1) (commercial powder from DIC called Linablue G1) was dissolved in water at pH 2 and 500 ppm concentration, then 500 ppm of rosmaric acid was added to the solution, thus forming an complex.

The complex formation was indicated by small increase in absorption of solution (11% increase in absorption and a shift in absorption maximum from 598 nm to 608 nm). Finally the vial was then sealed and placed in accelerated storage for 5 days. After the storage period the absorption was measured.

This sample lost 35% of its color strength.

### Example 11

Cleaved phycocyanin (see example 1) (commercial powder from DIC called Linablue G1) was dissolved in water at pH 3.5 (3.5 is a common pH in a beverage) and 500 ppm concentration, then 100 ppm of tannic acid was added to the solution, thus forming an complex.

The complex formation was indicated by substantial increase in absorption of solution (84% increase in absorption and a shift in absorption maximum from 564 nm to 604 nm). After 5 days of storage we could not measure any color breakdown (See FIG. 1).

This was confirmed with visual inspection where no color fading could be detected.

### Example 12

Cleaved phycocyanin (see example 1) (commercial powder from DIC called Linablue G1) was dissolved in water at pH 3.5 (3.5 is a common pH in a beverage) and 500 ppm concentration, then 100 ppm of tannic acid was added to the solution, thus forming an complex.

The complex formation was indicated by substantial increase in absorption of solution (84% increase in absorption and a shift in absorption maximum from 564 nm to 604 nm) (FIG. 1).

After 14 days of storage the sample had lost 18% of its color.

It should be noted that 14 days of accelerated storage roughly translate to the amount of light that the beverage can be exposed to in 6 months of storage on a shelf under supermarket lighting. It should further be noted that even though the color strength is measured 18% lower than in the beginning of the experiment, this small amount of reduction in color strength could not be noticed by visual comparison between fresh sample and stored sample (14 days of accelerated storage).

The color strengthen of the complex according to the invention decreased less or increased compared to blank or Gallic acid because of the polyphenol-chromophore-complex. The results are summarized in table 1 and FIG. 1.

TABLE 1
Overview over the examples.

| | | | | Additional | | | |
|---|---|---|---|---|---|---|---|
| | Linablue | | | ingredient | | Accelerated | Color |
| Example | concentration | Phycocyanin | Additional | concentration | | storage | loss |
| nr | (ppm) | treatment | ingredient | (ppm) | pH | (Days) | (%) |
| 3 | 500 | No treatment | No added ingredient | 0 | 2 | 5 | 100 |
| 4 | 500 | HCl treatment | No added ingredient | 0 | 2 | 5 | 100 |
| 5 | 500 | No treatment | No added ingredient | 0 | 3.5 | 5 | 100 |
| 6 | 500 | HCl treatment | No added ingredient | 0 | 3.5 | 5 | 100 |
| 7 | 500 | No treatment | Tannic acid | 100 | 2 | 5 | 63.9 |
| 8 | 500 | HCl treatment | Tannic acid | 100 | 2 | 5 | 35.1 |
| 9 | 500 | HCl treatment | Gallic acid | 500 | 2 | 5 | 100 |
| 10 | 500 | HCl treatment | Rosmarinic acid | 100 | 2 | 5 | 35 |
| 11 | 500 | HCl treatment | Tannic acid | 100 | 3.5 | 5 | 0 |
| 12 | 500 | HCl treatment | Tannic acid | 100 | 3.5 | 14 | 18.1 |

1.-15. (canceled)

16. A complex of at least one phycocyanobilin and at least one polyphenol obtainable by mixing the polyphenol with a composition comprising at least one phycocyanobilin in an aqueous solution.

17. The complex of claim 16, which is a stable, in water not dissociating complex.

18. The complex of claim 16, which is stable at pH 1 to 8.

19. The complex of claim 16, which has an increased absorption of light compared with pure phycocyanin with a wavelength within the interval from 550 to 670 nm.

20. The complex of claim 16, wherein the composition comprising at least one phycocyanobilin is obtained by cleaving phycocyanin.

21. The complex of claim 16, wherein the polyphenol is selected from the group of compounds comprising a least two phenol rings, each of them substituted with at least one hydroxy-groups and/or the polyphenol comprises a least one carboxylate ester group and/or carboxylic acid group optionally cycloalkyls or hetero-cycloalkyls with a carbon chain of C5 or C6.

22. The complex of claim 16, wherein the polyphenol is selected from the group of compounds comprising a least two phenol rings, each of them substituted with at least one hydroxy-groups and/or the polyphenol comprises a least one glucose group.

23. The complex of claim 16, comprising a protein which has a higher isoelectrical point than the phycocyanin, and/or polymer.

24. A process for formation of a complex comprising the step of mixing a polyphenol with a composition comprising at least one phycocyanobilin in an aqueous solution.

25. The process of claim 24, wherein the composition comprising at least one phycocyanobilin is obtained by cleaving phycocyanin by chemical and/or enzymatic cleavage.

26. The process of claim 25, wherein phycocyanin is cleaved by proteolysis.

27. The process of claim 26, wherein phycocyanin is cleaved by action of at least one strong acid and optionally heat.

28. The process of claim 24, wherein the solution of phycocyanin-fragments is spray-dried and redissolved in water.

29. The process of claim 24, comprising a step of adding a protein which has a higher isoelectrical point than the phycocyanin.

30. A food, feed, cosmetic or pharmaceutical preparation which comprises the complex of claim 16.

31. A colorant which comprises the complex of claim 16.
